(19) United States
(12) Patent Application Publication
(10) Pub. No.: US 2015/0305626 A1
(43) Pub. Date: Oct. 29, 2015

(54) DRIVE NOISE TOLERANT PLAQUE DETECTION

(71) Applicant: KONINKLIJKE PHILIPS N.V., Eindhoven (NL)

(72) Inventors: STEVEN CHARLES DEANE, CAMBRIDGE (GB); ALAN JAMES DAVIE, CAMBRIDGE (GB)

(21) Appl. No.: 14/442,813

(22) PCT Filed: Dec. 19, 2013
(86) PCT No.: PCT/IB2013/061122

§ 371 Date: May 14, 2015

Related U.S. Application Data
(60) Provisional application No. 61/739,512, filed on Dec. 19, 2012.

(51) Int. Cl. A61B 5/00; A46B 15/00; A61C 1/07

(52) U.S. Cl. CPC A61B 5/0088; A61B 5/0071; A61C 1/07; A61B 5/7282; A46B 15/0044; A61B 2576/02

(57) ABSTRACT

A dental implement (4) is presented that includes, in one embodiment, a light source (331) configured to emit an excitation light, and at least one optical unit for removing reflected excitation light and receiving a fluorescence light beam from the teeth. The dental implement further includes a detector (333) configured to receive the fluorescence light beam for detecting plaque and communicating a plaque identification signal of the teeth based on frequency domain lifetime measurements via a plaque detection circuit configured to reduce noise generated by a drive train at a drive train frequency (f_{dt}). The plaque identification signal is demodulated to an intermediate frequency (f_{IF}) such that the drive train frequency and the intermediate frequency are derived from a same master clock.

### TECHNICAL FIELD

The present disclosure relates to dental cleaning implements, such as toothbrushes. More particularly, the present disclosure relates to an electronic toothbrush for detecting plaque based on time resolved fluorescence, and in particular frequency domain lifetime measurements, as e.g. discussed in co-pending application U.S. 61/739,415 (Attorneys' reference 2012PF02051), incorporated herein by reference.

### BACKGROUND ART

Toothbrushes are designed to clean teeth by removing bio-films and food debris from teeth surfaces and interproximal regions in order to improve oral health. A wide variety of electronic toothbrush designs have been created to provide improved brushing performance by increasing the speed of the brush head and using sonic vibration, and in some cases ultrasonic vibration. Modern toothbrushes are very efficient at removing plaque. The consumer need only brush in the problem area for a few seconds to lift off plaque that is being brushed. However, without feedback the consumer may move on to another tooth before plaque has been completely removed. Thus, an indication of plaque levels on the teeth is highly desirable.

Despite improvements in toothbrush designs, an issue still remains in that a plaque detection circuit must be operated in close proximity to the toothbrush drive train, in the case of incorporation into an electronic toothbrush. This drive train emits electromagnetic interference, which may couple into the plaque detection circuits and inhibit the desired signal.

### SUMMARY OF THE INVENTION

Therefore, there is an increasing need to develop dental cleaning implements that may identify plaque and reduce signal interference between the plaque detection circuitry and the drive train signals. The invention is defined by the independent claims; the dependent claims define advantageous embodiments.

In one embodiment of the invention, a dental implement is provided that comprises:

a drive train having a drive train frequency;

a light source configured to emit an excitation light; and

a plaque detector configured to receive a return light beam from the teeth for generating a plaque identification signal, wherein the plaque identification signal is demodulated to an intermediate frequency such that the drive train frequency and the intermediate frequency are derived from a same master clock.

In accordance with other aspects of the present disclosure, a dental implement is presented. The dental implement includes an oscillator, a light source modulated by the oscillator and configured to emit an excitation light, and at least one optical unit for optionally removing reflected excitation light and receiving a fluorescence light beam from the teeth. The dental implement further includes a detector configured to receive the fluorescence light beam for detecting plaque and communicating a plaque identification signal of the teeth based on frequency domain lifetime measurements via a plaque detection circuit configured to reduce noise generated by a frequency drive train. The plaque identification signal is demodulated to a low intermediate frequency (IF) such that the drive train frequency and the intermediate frequency are derived from a same master clock.

The term “demodulation” refers to the extraction of an information bearing signal from a modulated carrier. In this case, the information results for example from fluorescent lifetime effects, which modify the phase and amplitude of the received signal.

The intermediate frequency may be equal to the drive train frequency multiplied by (n+j/k), where “n” is an integer, “j” is a positive integer, and “k” is an even positive integer such that 0<j<k.

“k” may be chosen such that a bandwidth of the plaque identification signal is less than a frequency of the drive train divided by “k.”

The demodulated intermediate frequency signal may be digitized at a frequency that is an integer multiplier at least twice its frequency.

The drive train frequency may be varied based on a plurality of cleaning modes.

A controller may drive the oscillator. A master clock of the controller may be an integer ratio of a master clock used for frequency modulation.

The dental implement may further include an optical excitation cleanup filter.

The at least one optical unit may be at least one of a long pass beam splitter, a short pass beam splitter, a bandpass filter, a band reject filter, a long pass filter, and a dichroic beam splitter.

According to yet another aspect of the disclosure, demodulation to the low intermediate frequency IF prevents harmonic interference with the plaque identification signal.

Another embodiment of the invention refers to a method of detecting plaque on teeth via a dental implement having a drive train having a drive train frequency, the method comprising the following steps:

providing an excitation light;

receiving a return light beam from the teeth;

generating a plaque identification signal from the return light beam; and

demodulating the plaque identification signal to an intermediate frequency such that the drive train frequency and the intermediate frequency are derived from a same master clock.

The method and the dental implement described above are related to each other such that embodiments and/or features explained for one of them are analogously valid for the other, too.

According to yet a further aspect of the disclosure, a method of detecting plaque on teeth via a dental implement is presented. The method includes the steps of providing an oscillator, providing a light source modulated by the oscillator and configured to emit an excitation light, optionally removing reflected excitation light via at least one optical unit, and receiving a fluorescence light beam from the teeth, via a detector. The method also includes the step of detecting plaque and communicating a plaque identification signal of the teeth based on frequency domain lifetime measurements via a plaque detection circuit configured to reduce noise generated by a frequency drive train. The method further includes the step of demodulating the plaque identification signal to a low intermediate frequency (IF) such that the drive train frequency and the intermediate frequency are derived from a same master clock.

Further scope of applicability of the present disclosure will become apparent from the detailed description given hereinafter.

### BRIEF DESCRIPTION OF THE DRAWINGS

The aspects of the present disclosure may be better understood with reference to the following figures illustrating embodiments. The components in the figures are not necessarily to scale, emphasis instead being placed upon clearly illustrating the principles of the disclosure. Moreover, in the figures, like reference numerals designate corresponding parts throughout the several views.

In the figures:

FIGS. 1A and 1B are front and side views, respectively, of a dental apparatus;

FIG. 2A illustrates a drive train driven at a first frequency;

FIG. 2B illustrates a drive train driven at a second frequency;

FIG. 3 is a circuit diagram of a plaque detection circuit;

FIG. 4 is a schematic diagram illustrating a plaque detection technique based on a fluorescence lifetime measurement, where a single detector is shown;

FIG. 5 is a schematic diagram illustrating a plaque detection technique based on a fluorescence lifetime measurement, where two detectors are shown;

FIG. 6 is a schematic diagram illustrating a plaque detection technique based on a fluorescence lifetime measurement, where an oscillator is incorporated within the controller; and

FIG. 7 is a flowchart illustrating a method of detecting plaque based on a fluorescence lifetime measurement.

### DETAILED DESCRIPTION OF EMBODIMENTS

The present disclosure describes various embodiments of systems, devices, and methods for helping users clean their teeth, in particular, by informing users whether they are indeed removing plaque from their teeth and if they have fully removed the plaque, providing both reassurance and coaching the users into good habits. Preferably the information is provided in real-time during brushing/cleaning, otherwise consumer acceptance is likely to be low. For example, it is useful for a dental implement (e.g., a toothbrush or airfloss) to provide the user with a signal when the tooth the user is brushing is considered clean, so that the user may move on to the next tooth, which may require additional brushing/cleaning due to plaque build-up. This may reduce the user's brushing/cleaning time, but also leads to a better and more efficient brushing/cleaning routine that focus the user's attention to specific problem areas of the teeth (e.g., that have plaque).

In accordance with the present disclosure, a user is able to detect plaque with an electronic dental cleaning implement, i.e., in a vibrating brushing/cleaning system surrounded with toothpaste foam. The plaque detection system is configured to provide a clear contrast between a surface with the removable plaque layers and a cleaner pellicle/calculus/dental filling/tooth surface.

In accordance with the present disclosure, there is provided a way to detect plaque in real-time during the brushing/cleaning routine. The exemplary embodiments of the present disclosure implement plaque detection based on time resolved fluorescence.

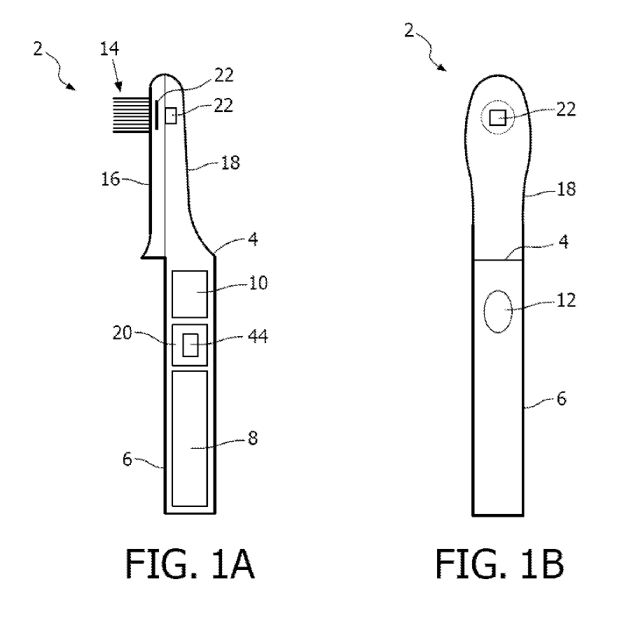

FIG. 1A illustrates a system 2 that is configured to detect dental plaque. System 2 may be configured for use with a variety of handheld dental implements. In the illustrated embodiment, system 2 is in the form of a multipurpose dental apparatus 4 (e.g., a combination electric toothbrush and dental plaque detector). Dental apparatus 4 includes a handle 6 of suitable configuration that is configured to house a battery 8 and an electric motor 10. A power button or switch 12 (FIG. 1B) is provided on the handle 6 and operably couples to battery 8 for supplying power to dental apparatus 4 and components operably associated therewith, e.g., electric motor 10, a controller 20, etc., when depressed. A plurality of bristles 14 of suitable configuration is provided on toothbrush assembly 16 that is configured to detachably couple via one or more coupling methods, e.g., clips (not explicitly shown), to a shaft 18 that extends distally from handle 6.

The apparatus 4 further comprises a plaque detection circuit 22 for indicating of plaque levels on the teeth. Such a plaque detection circuit must be operated in close proximity to the toothbrush drive train (with motor 10). This drive train emits electromagnetic interference, which may couple into the plaque detection circuits and inhibit the desired signal. While careful shielding, placement, and layout of the detection circuits may help to reduce these effects, it is useful to design a circuit that is inherently tolerant of the interference, thus enabling the best operation of the toothbrush. The problem is made more difficult, as the frequency of the drive train is varied between different modes of brushing, and many harmonics are produced depending on the duty cycle of the drive train excitation.

In accordance with the present disclosure, demodulation of a detected plaque signal is mixed down to a low, but non-zero intermediate frequency (IF). This prevents unwanted DC, generated by the unintentional leakage of the oscillator signal in the mixing process, causing an offset in the plaque detection circuit. This signal is then digitized and the desired signal is recovered from the data by signal processing techniques. A noise tolerant system is accomplished by deriving the drive train, modulation, ADC sampling, and intermediate frequency from the same master clock, and making the intermediate frequency equal to the drive train frequency multiplied by (n+j/k), where “n” is an integer and may be zero, “k” is an even positive integer, and “j” is a positive integer such that 0<j<k. Further, “k” must not be too large or signal interference may occur. Thus, “k” is chosen such that the bandwidth of the plaque detection signal is less than the frequency of the drive train divided by “k.”

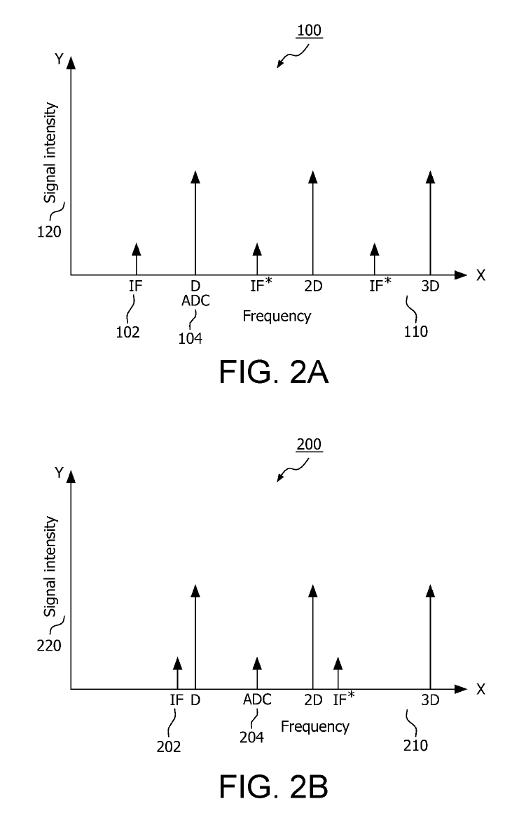

FIG. 2A illustrates a drive train 100 driven at a first frequency.

With reference to FIG. 2A, demodulation of a detected plaque signal is mixed down to a low, but non-zero intermediate frequency (IF). This prevents DC from the oscillator from causing an offset in the plaque detection circuit. This signal is then digitized and the desired signal is recovered from the data by signal processing techniques. A noise tolerant system is accomplished by deriving the drive train, modulation, ADC sampling, and IF from the same master clock, and making the IF frequency equal to the drive train frequency multiplied by (n+j/k), where “n” is an integer and may be zero, “k” is an even positive integer, and “j” is a positive integer such that 0<j<k. Further, “k” must not be too large or signal interference may occur. Thus, “k” is chosen such that the bandwidth of the plaque detection signal is less than the frequency of the drive train divided by “k.”

In particular, where the multiplier is 0.5, it is seen that the drive train excites once (D, 2D, 3D, etc.) in each half of the IF cycle, and, thus, any interference from its fundamental or harmonics is canceled out. However, it may be desirable to make the IF higher to optimize circuit cost and performance, and by, for example, spectral analysis it is determined that any permitted value of “n,” “j,” and “k” results in the desired narrow band signal being optimally spaced between the harmonics of the drive train interference, and so recovered by narrow band filtering either in the digital or analog domain. In FIG. 2A, the x-axis 110 is represented as frequency and the y-axis 120 is represented as signal intensity. The IF is depicted on the left hand side of the x-axis 110 as element 102. The point at which ADC conversion occurs is depicted as element 104 and occurs after the first instance of the IF 102. The drive train 100 in FIG. 2A, is considered a simple case, where n=0, j=1, and k=2, which places the images of the signal of interest exactly between the harmonic noise of the drive train 100. In general, j=1, k=2 provides the best spacing margin, and, thus, the lowest filtering requirements for any “n.” However, one skilled in the art may contemplate any values for “j” and “k” to minimize the noise of the drive train 100.

FIG. 2B illustrates a drive train 200 driven at a second frequency.

In FIG. 2B, the x-axis 210 is represented as frequency and the y-axis 220 is represented as signal intensity. The IF is depicted on the left hand side of the x-axis 210 as element 202. The point at which ADC conversion occurs is depicted as element 104 and occurs well after the first instance of the IF 202. The multiplier is 0.75, and it is seen that the drive train no longer excites twice (D, 2D, 3D, etc.) in each half of the IF cycle, yet non-interference of the harmonics is maintained. The drive train 200 in FIG. 2B, is a complex case, where n=0, j=3, k=4, which places the images of the signal of interest away from the harmonic noise of the drive train, with enough margin to avoid interference. While this solution is workable, it has more stringent filtering requirements, as the spacing between the desired signal and the drive train harmonics is reduced. Once again, one skilled in the art may contemplate any values for “j” and “k” to minimize the noise of the drive train 200.

With reference to FIGS. 2A and 2B, it is noted that one skilled in the art may contemplate using a plurality of different multipliers in order to reduce the harmonics, and thus, reduce the interference between the plaque detection circuitry and the drive train signal.

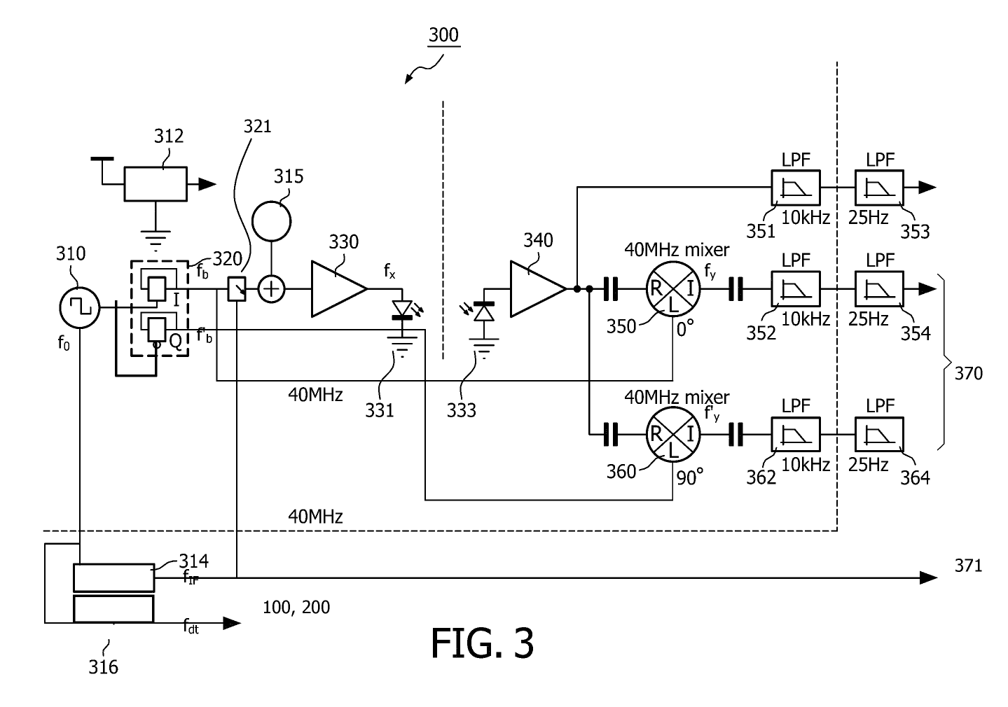

FIG. 3 is a circuit diagram 300 of a plaque detection circuit.

The circuit 300 includes an oscillator 310 and a voltage regulator 312. The oscillator 310 provides a master clock signal of frequency f₀, for example of 80 MHz. The frequency f₀ of the oscillator 310 is received by element 320. Element 320 is circuitry that divides the fundamental clock down (e.g. by the factor ½) to generate a base clock signal of frequency f_{b} and a 90 degree shifted phase clock signal of frequency f_{b}, with the base signal f_{b} being used to modulate the excitation light, and both being used for the phase detection. The frequency f_{b} of the base signal may for example be 40 MHz.

The base signal f_{b} is provided to a multiplier (or mixer) 321 which additionally receives a signal of an intermediate frequency f_{IF}. The output signal of the multiplier 321 has frequencies (f_{b}+F_{IF}) and (f_{b}−f_{IF}), briefly denoted as fₓ in the Figure (it should be noted that fₓ comprises both frequencies, from which the original IF signal is recovered in the mixer(s) on the detector side).

An LED driver 330 is powered by a DC bias 315. The LED driver 330 drives the excitation light source 331, which is modulated by the output signal fₓ of the multiplier 321. The light emitted from the excitation light source 331 and particularly fluorescent light excited by this excitation light when shining on the teeth is detected by the photodetector 333 connected to the amplifier 340.

The output of the amplifier 340 is received by a first mixer 350, a second mixer 360, and a low-pass filter 351. By multiplying by the f_{b} signal, the first mixer 350 produces a signal at the intermediate frequency f_{IF}. By multiplying by the 90° shifted signal f_{b}, the second mixer 360 produces a signal at the intermediate frequency f_{IF} related to the phase shifted component.

The output of the first mixer 350 is received by a first series of low-pass filters 352, 354, whereas the output of the second mixer 360 is received by a second series of low-pass filters 362, 364. The output of the low-pass filter 351 is received by another low-pass filter 353. The output signals of the low pass filters 353, 354, and 364 are further processed in a subsequent stage 370 of the plaque detection circuit to detect plaque based on frequency domain fluorescence lifetime measurements.

Element 314 generates the IF signal of frequency f_{IF} from the master clock f₀, and uses it to both modulate the excitation light (via multiplier 321) and to digitally lock in to the digitized signal (in a microprocessor 371).

Element 316 generates the master clock for the drive train 100 (or 200), having drive train frequency f_{dt}. The drive train frequency f_{dt} is based on the master clock frequency f₀, but typically will be divided down e.g. from 40 MHz to f_{dt}=250 Hz. By being based on the same master clock, the phase lock and exact ratio are guaranteed. Elements 314 and 316 work together to maintain the fixed relationship between their output frequencies given by the formula: f_{IF}=(n+j/k)·f_{dt}. As a result, this circuit design minimizes the susceptibility of the plaque detection circuit to noise and maximizes the accuracy of the levels of plaque detection detected by the plaque detection circuit. Thus, even if low levels of plaque are found on the teeth, the plaque detection circuit has the capability of such low-detection because the noise generated by the drive train is minimized by the techniques of the present exemplary embodiments.

Referring again to FIG. 3, as an example, if the drive train excitation frequency is 250 Hz, an IF of f_{IF}=875 Hz (n=3, multiplier 3.5) or 1125 Hz (n=4, multiplier 4.5) may be convenient. If in a different mode the drive train were driven at 260 Hz, then an IF of 910 or 1170 Hz may be chosen. When the drive train frequency is changed, the multiple of the signal used for the IF need not stay the same. For example, if a significant shift of drive frequency is used, the multiple or multiplier may change to avoid the IF becoming an inconveniently high or low value.

The demodulated intermediate frequency signal (f_{y}, f′_{y}) may be digitized at a frequency which is an integer multiplier, at least twice its frequency to capture all its information according to the Nyquist criterion. This frequency changes as the drive train excitation frequency and IF change. However, once again, the multiplier need not be fixed if desired to maintain optimum ADC performance in all modes.

In another exemplary embodiment, the drive train signal is further derived from an integer division of the modulation frequency. In practice, due to the large differences, this is not significantly limiting on the choice of drive train frequencies, as typically the drive train may be at 260 Hz, while the modulation may be at 40 MHz.

Similarly, if digital circuits are used, such as a microprocessor to perform digital signal processing, its master clock should be an integer multiple or divisor of the master clock (f₀) used for the modulation. This further minimizes interference between the plaque detection circuitry and the drive train signal. As a result, in accordance with FIGS. 1-3, all these steps prevent unwanted harmonic interference from the various components resulting in a signal that interferes with the plaque detection circuit.

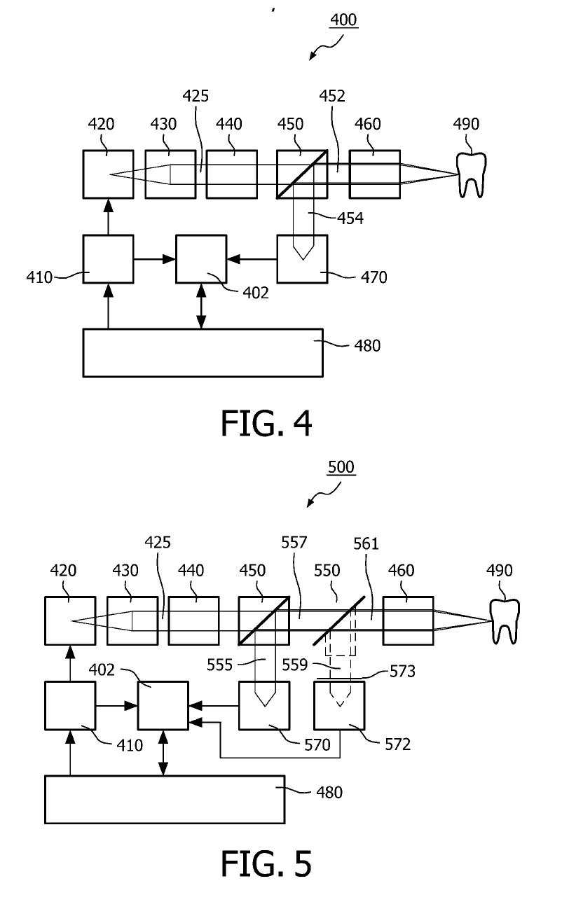

FIG. 4 depicts a schematic diagram 400 illustrating a plaque detection technique based on a fluorescence lifetime measurement, where a single detector is shown.

In FIG. 4, an oscillator 410 is depicted for driving a light source 420. The light source 420 generates an excitation light 425 passing through a first optical element configuration 430, a cleanup filter 440, and a beam splitter 450. The beam splitter 450 allows the excitation light 425 to pass straight through. The returning light 452 from the teeth 490 is split so that only the fluorescent light 454 is received by the detector 470. The excitation light 425 is directed through a second optical element configuration 460 and onto teeth 490, whereas the fluorescent light 454 is directed toward a detector 470. The detector 470 may include an amplifier 402. It is also contemplated that the oscillator 410 is driven by a controller 480.

The light source 420 generating the excitation light 425 is preferably an LED of 405 nm, 440 nm, 470 nm or 480 nm, but other sources (e.g., diode laser) and different wavelengths are also possible (e.g., ranging between about 400 nm and 500 nm). One skilled in the art may contemplate a plurality of different lighting means operating at a plurality of different wavelengths. The diode laser may also be a vertical cavity surface emitting laser (VCSEL). The VCSEL is a type of semiconductor laser diode with laser beam emission perpendicular from the top surface, contrary to conventional edge-emitting semiconductor lasers, which emit from surfaces formed by cleaving the individual chip out of a wafer.

The optional cleanup filter 440 may be a narrow bandpass filter, which blocks any undesired wavelength from reaching the teeth 490 (e.g., UV light) or the detector 470. The dichroic beam-splitter 450 may have a short-pass characteristic, such that the excitation light 425 is transmitted towards the teeth 490, while the emitted fluorescence light 454, having a longer wavelength, is reflected towards the detector 470. The detector 470 may include a photodetector (not shown) and an amplifier 402. The system 400 may also include a collection of focusing optics, such as lenses, CPC's (compound parabolic concentrators) or both (shown as elements 430, 460). In one exemplary embodiment, the optical elements 430, 460 of the system may be integrated into the head portion of the dental implement. However, one skilled in the art may contemplate rearranging or placing all or part of the elements of FIG. 4 either in the handle portion or the head portion of the dental implement or a combination thereof based on suitable designs. Thus, the components of FIG. 4 are not limited as to their placement on or about a dental implement.

Moreover, in another exemplary embodiment, instead of a single optical path and a beam splitter, two optical paths (e.g., excitation and detection) may be used with a high pass or bandpass or band-reject filter at the detector 470 to block the excitation light 425. The separate excitation and detection paths may be fiber guided or free space or a combination of both, e.g., free-space excitation via an LED in the brush-head and fiber detection.

The controller 480 can be a processor, microcontroller, a system on chip (SOC), field programmable gate array (FPGA), etc. Collectively the one or more components, which can include a processor, microcontroller, SOC, and/or FPGA, for performing the various functions and operations described herein are part of a controller, as recited, for example, in the claims. The controller may be provided as a single integrated circuit (IC) chip which may be mounted on a single printed circuit board (PCB). Alternatively, the various circuit components of the controller, including, for example, the processor, microcontroller, etc. are provided as one or more integrated circuit chips. That is, the various circuit components are located on one or more integrated circuit chips.

FIG. 5 depicts a schematic diagram 500 illustrating a plaque detection technique based on a fluorescence lifetime measurement, where two detectors are shown.

In FIG. 5, an oscillator 410 is depicted for driving a light source 420. The light source 420 generates an excitation light 425 passing through a first optical element configuration 430, a cleanup filter 440, and two beam splitters 450, 550. The beam splitters 450, 550 allow the excitation light 425 to pass straight through. Thus, the excitation light 425 passes straight through the two beam splitters 450, 550 and excites the fluophores on the teeth and plaque. The light 561 returned from the teeth 490 and collected by the system consists of reflected excitation light (blue) 557 and fluorescence light 555 (emission with longer wavelength). On the way back into the system 500, it first reaches the low reflection beam splitter (glass) to couple out a small fraction. The remainder goes to the beam splitter 450 and towards the detector 570. The fractional part coming from the glass reflection is short (or band-) pass filtered such that only the original excitation light 559 is detected by 572. This is referred to as the reference signal.

FIG. 5 is similar to FIG. 4, however, in FIG. 5, a portion of the reflected excitation light 561 is measured separately to compensate for any drift which may cause undesired phase changes between excitation and emission signals, for example, an optical path length difference caused by distance variations or temperature effects. The extension uses a low reflection beam splitter 550 (e.g., uncoated glass) to couple out a low percentage of the received light 461. A low pass filter 573 removes the fluorescence light such that only part of the reflected excitation light 561 is received by the detector 572. This light 559 has travelled the full path length and is therefore a reference for phase.

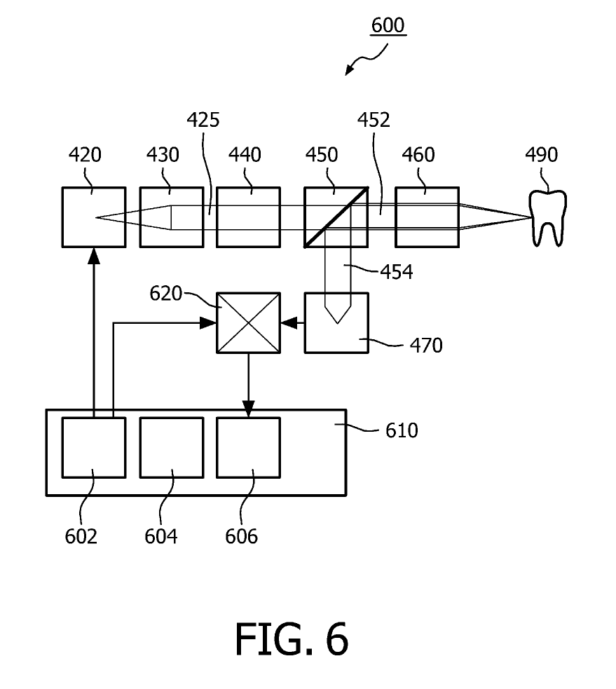

FIG. 6 depicts a schematic diagram 600 illustrating a plaque detection technique based on a fluorescence lifetime measurement, where an oscillator is incorporated within the controller.

In FIG. 6, an oscillator 602 is depicted for driving a light source 420. The light source 420 generates an excitation light 425 passing through a first optical element configuration 430, a cleanup filter 440, and a beam splitter 450. The beam splitter 450 allows the excitation light 425 to pass straight through. The returning light 452 from the teeth 490 is split so that only the fluorescent light 454 is received by the detector 470. The excitation light 425 is directed through a second optical element configuration 460 and onto teeth 490, whereas the fluorescent light 454 is directed toward a detector 470. The detector 470 sends a signal to a mixer 620. It is also contemplated that the oscillator 602 is incorporated within a controller 610. The controller 610 may also include a lock-in amplifier 604 and an ADC converter 606.

For all embodiments, as described with reference to FIGS. 4-6, the oscillator 610 and lock-in amplifier 604 may be implemented in the analog or digital domain. However, in accordance with FIG. 6, for the digital implementation into the controller 610, an analog heterodyning stage may be included to down convert the signals to an intermediate frequency (IF) band that is better suited for ADC conversion. Therefore, for each frequency, the digital oscillator 602 further generates a frequency with a small offset for the mixer 620, such that the low-pass filtered mixer 620 output falls within the frequency range of the ADC converter 606. In such case, all further signal processing is performing in a digital manner. Moreover, even though FIG. 6 describes the digital implementation for only one of the embodiments, it should be noted that all embodiments may be implemented this way by one skilled in the art.

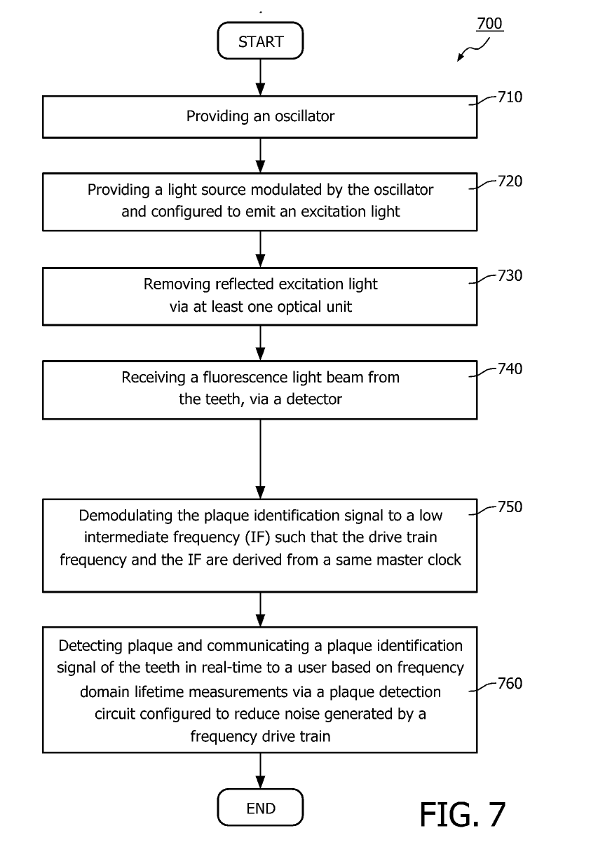

FIG. 7 is a flowchart 700 illustrating a method of detecting plaque based on a fluorescence lifetime measurement.

The flowchart 700 includes the following steps. In step 710, an oscillator is provided. In step 720, a light source modulated by the oscillator and configured to emit an excitation light is provided. In optional step 730, reflected excitation light is removed via at least one optical unit. In step 740, a fluorescence light beam is received from the teeth via a detector. In step 750, the plaque identification signal is demodulated to a low intermediate frequency (IF) such that the drive train frequency and the IF are derived from a same master clock. In step 760, plaque is detected and a plaque identification signal of the teeth is communicated in real-time to a user based on frequency domain lifetime measurements via a plaque detection circuit configured to reduce noise generated by a frequency drive train. The process then ends. It is to be understood that the method steps described herein need not necessarily be performed in the order as described. Further, words such as “thereafter,” “then,” “next,” etc. are not intended to limit the order of the steps. These words are simply used to guide the reader through the description of the method steps.

In general, the exemplary embodiments of the present disclosure specifically relate to dental cleaning implements, such as toothbrushes or airfloss devices, as well as professional dental examination devices, whereby presence of plaque may be revealed by images, sound or vibration frequency and intensity. This is applicable in fields such as dentistry, dental hygiene, and tooth whitening.

The foregoing examples illustrate various aspects of the present disclosure and practice of the methods of the present disclosure. The examples are not intended to provide an exhaustive description of the many different embodiments of the present disclosure. Thus, although the foregoing present disclosure has been described in some detail by way of illustration and example for purposes of clarity and understanding, those of ordinary skill in the art will realize readily that many changes and modifications may be made thereto without departing form the scope of the present disclosure as defined by the independent claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The invention may be implemented by means of hardware comprising several distinct elements, and/or by means of a suitably programmed processor. In the device claim enumerating several means, several of these means may be embodied by one and the same item of hardware. The mere fact that certain measures are recited in mutually different dependent claims does not indicate that a combination of these measures cannot be used to advantage.

1. A dental implement, comprising:
a drive train having a drive train frequency (f_{dt});
a light source configured to emit an excitation light; and
a plaque detector configured to receive a return light beam from the teeth for generating a plaque identification signal;
wherein the plaque detector demodulates identification signal to an intermediate frequency (f_{IF}) such that the drive train frequency (f_{dt}) and the intermediate frequency (f_{IF}) are derived from a same master clock (f₀).

2. The dental implement according to claim 1, further comprising an oscillator for modulating the light source.

3. The dental implement according to claim 1 or the method, wherein the return light is fluorescence light.

4. The dental implement according to claim 3, further comprising at least one optical unit for removing reflected excitation light and receiving a fluorescence light beam from the teeth,
wherein the plaque detector is configured to receive the fluorescence light beam for detecting plaque and communicating a plaque identification signal of the teeth based on frequency domain lifetime measurements via a plaque detection circuit configured to reduce noise generated by a frequency drive train.

5. The dental implement according to claim 1, wherein the intermediate frequency (f_{IF}) is equal to the drive train frequency (f_{dt}) multiplied by (n+j/k), where “n” is an integer, “j” is a positive integer, and “k” is an even positive integer such that 0<j<k.

6. The dental implement according to claim 5, wherein “k” is chosen such that a bandwidth of the plaque identification signal is less than a frequency of the drive train divided by “k.”

7. The dental implement according to claim 1, wherein the plaque detector digitizes the demodulated intermediate frequency signal at a frequency that is an integer multiplier at least twice its frequency.

8. The dental implement according to claim 1, wherein the drive train varies the drive train frequency (f_{dt}) for a plurality of cleaning modes.

9. The dental implement according to claim 1, wherein an oscillator is the source used to generate the excitation light.

10. The dental implement according to claim 1, wherein the plaque detector demodulates the plaque identification signal to the intermediate frequency (f_{IF}) to prevent harmonic interference with the plaque identification signal.

11. A method of detecting plaque on teeth via a dental implement having a drive train having a drive train frequency (f_{dt}), the method comprising:
providing an excitation light;
receiving a return light beam from the teeth;
generating a plaque identification signal from the return light beam; and
demodulating the plaque identification signal to an intermediate frequency (f_{IF}) such that the drive train frequency (f_{dt}) and the intermediate frequency (f_{IF}) are derived from a same master clock.

12. The method according to claim 11, wherein the intermediate frequency (f_{IF}) is equal to the drive train frequency (f_{dt}) multiplied by (n+j/k), where “n” is an integer, “j” is a positive integer, and “k” is an even positive integer such that 0<j<k.

13. The method according to claim 12, wherein “k” is chosen such that a bandwidth of the plaque identification signal is less than a frequency of the drive train divided by “k.”

14. The method according to claim 11, further comprising digitizing the demodulated intermediate frequency signal at a frequency that is an integer multiplier at least twice its frequency.

15. The method according to claim 11, further comprising varying the drive train frequency (f_{dt}) based on a plurality of cleaning modes.
